(19) United States
(12) Patent Application Publication
(10) Pub. No.: US 2022/0033747 A1
(43) Pub. Date: Feb. 3, 2022

(54) Cell Culture Sensor Insert System

(71) Applicant: Scientific Industries, Inc., Bohemia, NY (US)

(72) Inventors: Joseph G. Cremonese, Greensburg, PA (US); E. Scott Anderson, Greensburg, PA (US); Jake Boy, Pittsburgh, PA (US); James Clancy, Blawnox, PA (US); Kenneth Kato, Export, PA (US); Doug Koebler, Irwin, PA (US); Richard Rodgers, Oakmont, PA (US)

(21) Appl. No.: 16/941,116

(22) Filed: Jul. 28, 2020

(51) Int. Cl. C12M 1/24; C12M 3/06; C12M 1/34

(52) U.S. Cl. CPC C12M 23/08; C12M 41/26; C12M 27/16

(57) ABSTRACT

Embodiments disclosed herein relate to novel apparatuses, methods and systems employing said novel apparatuses, and sensors for use with said apparatuses and systems. In some embodiments, apparatuses and systems described herein are used to culture cells and/or detect changes in pH levels or dissolved oxygen and/or carbon dioxide levels in a cell culture sample resulting from the growth of living cells contained within a reaction vessel such as a flask. In some embodiments, said novel apparatuses may be used in combination with a rocker or shaking device or apparatus. The systems and methods may be used to detect changes in pH and dissolved gas levels in a liquid contained in the reaction vessel due to growth of living cells.

### BACKGROUND OF THE INVENTION

### Field of the Invention

Embodiments disclosed herein relate to novel apparatuses, methods and systems employing said novel apparatuses, and sensors for use with said apparatuses and systems. In some embodiments, apparatuses and systems described herein are used to culture cells and/or detect changes in pH levels or dissolved oxygen and/or carbon dioxide levels in a cell culture sample resulting from the growth of living cells contained within a reaction vessel such as a flask. In some embodiments, said novel apparatuses may be used in combination with a rocker or shaking device or apparatus. The systems and methods may be used to detect changes in pH and dissolved gas levels in a liquid contained in the reaction vessel due to growth of living cells.

### Background of the Related Art

The first published record of cell culturing was made in 1885. Carrel, Burrows and Montrose, in 1910, performed noteworthy experiments by keeping mammalian tissue explants alive for two months. Then, from 1912 through 1946, Alexis Carrel, while working at Rockefeller University, made history by keeping embryo chick heart cells growing and dividing in the lab for more than thirty years. That, it seems, was the beginning of cell culturing as it is known today.

Advances in the development of cell culture growth vessels have been made, starting with the Carrel Flask and advancing through many iterations and modifications, mostly occurring from 1965 through 1995. Based on the growing knowledge of in-vitro cell culturing methods, the vessels changed from glass to newly developed plastics with various unique cell growth surface treatments. Since the beginning of such advancements in cell culture vessel design, certain of Carrel's goals and designs for the improvement of cell culturing were relegated to “the cutting room floor.”

Osmotic pressure control within modern cell culture vessels became an issue because of the increased vapor pressure at the surface of the liquid media at incubation temperature. T-flasks, as an example, in the effort to increase the cell growth surface and prolong culture times between media changes, became less efficient in preventing media desiccation. Increased quantity of nutrient medium became an increasing barrier to the effective transfer of oxygen from the “head-space” of the vessel to the cell growth layer.

Carbon dioxide build-up in the nutrient medium, a by-product of cell metabolism, became an increasing problem with the increasing growth rate of cells. Media formulations were buffered to inhibit the excess carbon dioxide build-up from lowering the pH of the nutrient medium and thereby harming the cells.

As methods and processes were modified to enhance in-vitro cell growth, the detrimental effects from increased cell growth rate became the most significant deterrent to improved cell growth. In-vitro cell culture became a dynamic paradox resulting from efforts to improve the process.

In-vitro cell culture processes have failed to keep up with the very rapid advancement of the understanding of in-vivo processes at the cellular and molecular levels. In-vitro cell culturing has stalled. Restarting the process and the development of the understanding of what can be done in vessel and process control changes and improvement will require a better understanding of the dynamics of in-vitro cell culturing. That will start with the ability to collect real-time information as the metabolic process of cell growth occurs in the cell culture environment.

Methods for monitoring and adjusting dissolved oxygen and carbon dioxide levels, measuring and maintaining pH stability and cell density and ensuring cell viability are first-line tools required to provide necessary diagnostic information.

To obtain such necessary diagnostic information, means of measuring dissolved gas and pH levels must be employed.

A significant challenge in obtaining necessary diagnostic information is the question of how to place necessary sensors into sterile cell culture vessels without contaminating the vessels or the environment within or surrounding the vessels.

It is therefore an object of apparatuses described herein for use in preparing and maintaining cell culture vessels, systems described herein comprising said apparatuses, and methods described herein of using said apparatuses to provide a means of culturing cells wherein the problem of contamination is eliminated.

Apparatuses, systems, and methods described herein permit the application of necessary sensors to a surface of the interior of a cell culture vessel with accuracy and precision and without risking contamination of the cell culture environment, regardless of the type, size or shape of the cell culture vessel employed.

### SUMMARY

Described herein are various, non-limiting embodiments of an apparatus for use in cell culturing comprising an elongated shaft having a proximal end and a distal end, a grippable portion at the proximal end of the elongated shaft, a threaded portion at the distal end of the elongated shaft, and a sensor frame having a threaded hole, wherein the threaded portion at the distal end of the elongated shaft is configured to be removably screwed into the threaded hole of the sensor frame.

Also described herein are various, non-limiting embodiments of a system for culturing cells comprising a flask, a detector system, at least one optical sensor operably connected to the detector system, and an apparatus comprising an elongated shaft having a proximal end and a distal end, a grippable portion at the proximal end of the elongated shaft, a threaded portion at the distal end of the elongated shaft, and a sensor frame having a threaded hole, wherein the threaded portion at the distal end of the elongated shaft is configured to be removably screwed into the threaded hole of the sensor frame, and wherein the at least one optical sensor is positioned within or on the sensor frame.

Furthermore, described herein are various, non-limiting embodiments of a method for culturing cells comprising placing an adhesive sheet on an underside of a sensor frame having a threaded hole, placing at least one optical sensor on or within the sensor frame, inserting the sensor frame into a flask such that the adhesive sheet makes contact with an interior surface of the flask and adheres to the interior surface; and placing the flask on or against a detector system, and culturing cells in the flask, wherein the at least one optical sensor is operably connected to the detector system, wherein the inserting is carried out using an apparatus, and wherein the apparatus comprises an elongated shaft having a proximal end and a distal end, a grippable portion at the proximal end of the elongated shaft, a threaded portion at the distal end of the elongated shaft, wherein the threaded portion at the distal end of the elongated shaft is configured to be removably screwed into the threaded hole of the sensor frame.

While these potential advantages are made possible by technical solutions offered herein, they are not required to be achieved. Embodiments of the presently disclosed apparatus, system and method can be implemented to achieve technical advantages, whether or not these potential advantages, individually or in combination, are sought or achieved.

Further features, aspects, objects, advantages, and possible applications of the present invention will become apparent from a study of the exemplary embodiments and examples described below, in combination with the Figures, and the appended claims.

### BRIEF DESCRIPTION OF THE DRAWINGS

The above and other objects, aspects, features, advantages and possible applications of the present invention will be more apparent from the following more particular description thereof, presented in conjunction with the following drawings, in which:

FIG. 1A depicts an embodiment of an exemplary sensor frame insertion apparatus described herein.

FIG. 1B depicts an embodiment of an exemplary sensor frame described herein.

FIG. 1C depicts an embodiment of an adhesive sheet to be applied to a sensor frame as described herein.

FIG. 1D depicts an embodiment of an exemplary sensor frame insertion apparatus and an exemplary sensor frame as described herein, wherein the threaded portion at the distal end of the sensor frame insertion apparatus is screwed into a threaded hole located on the sensor frame.

FIG. 2 depicts an embodiment of an exemplary sensor frame as described herein.

FIGS. 3A and 3B depict various views of an embodiment of the threaded portion of the distal end of an exemplary sensor frame insertion apparatus described herein.

FIGS. 4A and 4B depict views of an exemplary sensor frame insertion apparatus inserting an exemplary sensor frame into a flask according to a method described herein.

FIG. 4C depicts a sensor frame inserted into a flask according to a method described herein.

FIGS. 5A and 5B depict an exemplary sensor frame insertion apparatus having an adjustable ball stop positioned along the elongated shaft portion. FIGS. 5A and 5B show the adjustable ball stopper positioned at different positions along the elongated shaft portion.

FIGS. 6A and 6B depict an exemplary sensor frame insertion apparatus having an adjustable ball stop positioned along the elongated shaft portion which has been inserted into a flask. FIGS. 6A and 6B show the adjustable ball stopper positioned at different positions along the elongated shaft portion, resulting in different lengths of the elongated shaft portion being within the flask.

### DETAILED DESCRIPTION OF THE INVENTION

The following description is of an embodiment presently contemplated for carrying out the present invention. This description is not to be taken in a limiting sense, but is made merely for the purpose of describing the general principles and features of the present invention. The scope of the present invention should be determined with reference to the claims.

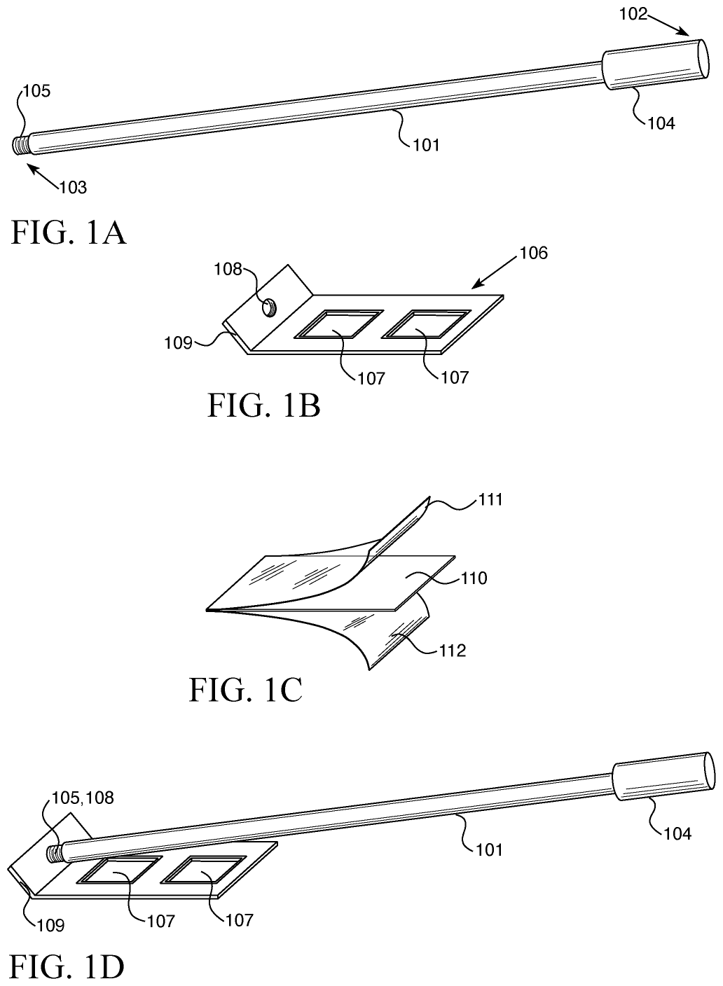

Referring to FIG. 1A, a sensor frame insertion apparatus described herein comprises an elongated shaft 101 having a proximal end 102 and a distal end 103.

Preferably, the sensor frame insertion apparatus comprises a grippable portion 104 at the proximal end 102 and a threaded portion 105 at the distal end 103.

Described herein are various, non-limiting embodiments of an apparatus for use in cell culturing comprising an elongated shaft having a proximal end and a distal end, a grippable portion at the proximal end of the elongated shaft, a threaded portion at the distal end of the elongated shaft, and a sensor frame having a threaded hole, wherein the threaded portion at the distal end of the elongated shaft is configured to be removably screwed into the threaded hole of the sensor frame.

Referring to FIG. 1B, a sensor frame described herein may comprise a flat or essentially flat sensor-housing body portion 106, which comprises one or more sensor-housing openings 107.

The sensor frame further comprises a threaded hole 108 configured to receive the threaded portion 105 of the distal end 103 of the elongated shaft 101 such that the threaded portion 105 may be removably secured into the threaded hole 108 using finger- or grip-tight strength.

In a preferred embodiment, the sensor frame comprises an angled portion 109, which extends upwardly from the sensor-housing body portion 106.

In an aspect, the angled portion 109 may be at any angle relative to the sensor-housing body portion 106. In an aspect, the angled portion 109 extends away from the sensor-housing body portion 106 at an angle of at least 90 degrees.

Although the figures are not to be interpreted as being to scale and are not intended to limit the scope of embodiments described herein, FIG. 1B shows, for example and by way of illustration, an angled portion 109 which extends away from the sensor-housing body portion at an angle of greater than 90 degrees.

Referring to FIG. 1C, an adhesive sheet 110 to be applied to a sensor frame described herein may have a first film 111 and a second film 112 which may be removed to expose a first adhesive surface and a second adhesive surface of the adhesive sheet 110, respectively.

In an aspect, one of the adhesive surfaces of the adhesive sheet 110 is to be applied to the underside of the sensor frame, in particular to the underside of the sensor-housing body portion 106, the underside being the surface of the sensor-housing body portion 106 which is further away from the sensor frame insertion tool when the sensor frame insertion tool is removably screwed into the threaded hole 108 of the sensor frame.

In another aspect, the other side of the adhesive sheet 110—that is, the surface of the adhesive sheet 110 which is not adhered to the underside of the sensor-housing body portion 106 of the sensor frame—is to be applied and adhered to an interior surface of a cell culture flask.

Referring to FIG. 1D, which depicts the sensor frame insertion apparatus removably screwed into the sensor frame via the threaded portion 105 of the insertion apparatus and the threaded hole 108 of the sensor frame, a skilled artisan may grip or otherwise hold the elongated shaft 101 portion via the proximal end 102, optionally and preferably via an optional grippable portion 104 located at the proximal end 102.

The skilled artisan may thus manipulate and position the sensor frame precisely while the sensor frame is being inserted into the interior of a cell culture flask. In an aspect, such a configuration of the sensor frame insertion apparatus and sensor frame, which may be removably secured together, a skilled artisan can carefully manipulate the sensor frame within 3D space inside a cell culture flask with precision so as to prevent contamination of the cell culture environment.

In certain embodiments, as depicted in FIGS. 1B and 1D, the threaded hole 108 may face toward the sensor-housing openings 107.

In an aspect, the upper-side of the adhesive sheet or adhesive film is partially exposed via one or more sensor-housing openings. In a further aspect, one or more sensors are adhered to the exposed portions of the adhesive sheet/film prior to insertion of the sensor frame into the cell culture flask.

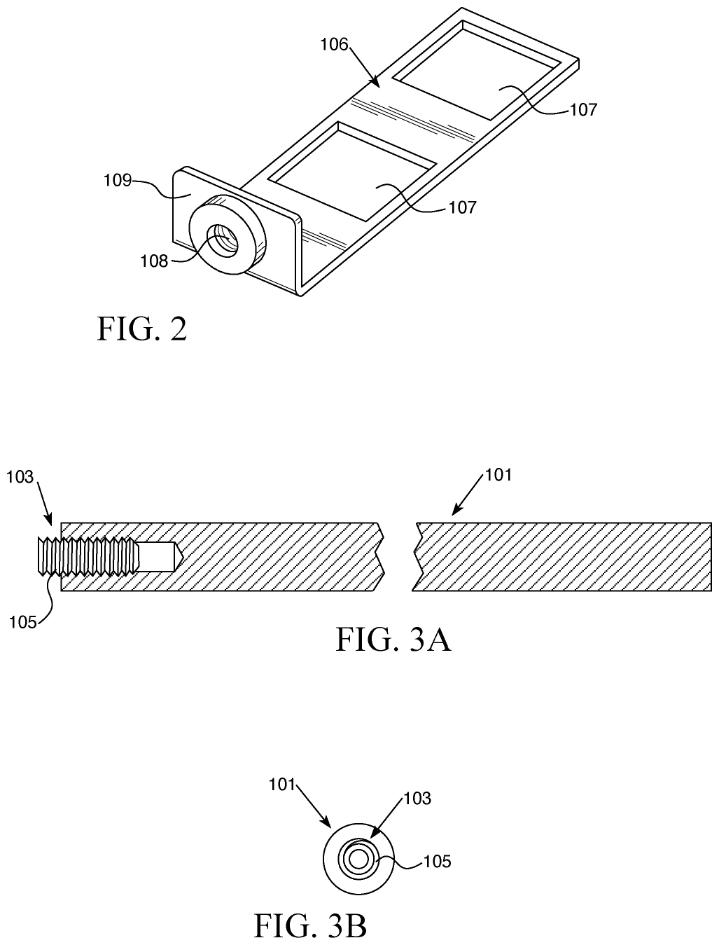

In other embodiments, such as the embodiment depicted in FIG. 2, the threaded hole 108 may face away from the sensor-housing openings 107.

FIG. 3A depicts a cross-sectional view of the threaded portion 105 in the distal end 103 of the elongated shaft 101 of the sensor frame insertion apparatus.

FIG. 3B depicts a head-on view of the distal end 103 of the elongated shaft 101 of the sensor frame insertion apparatus, including a head-on view of the threaded portion 105.

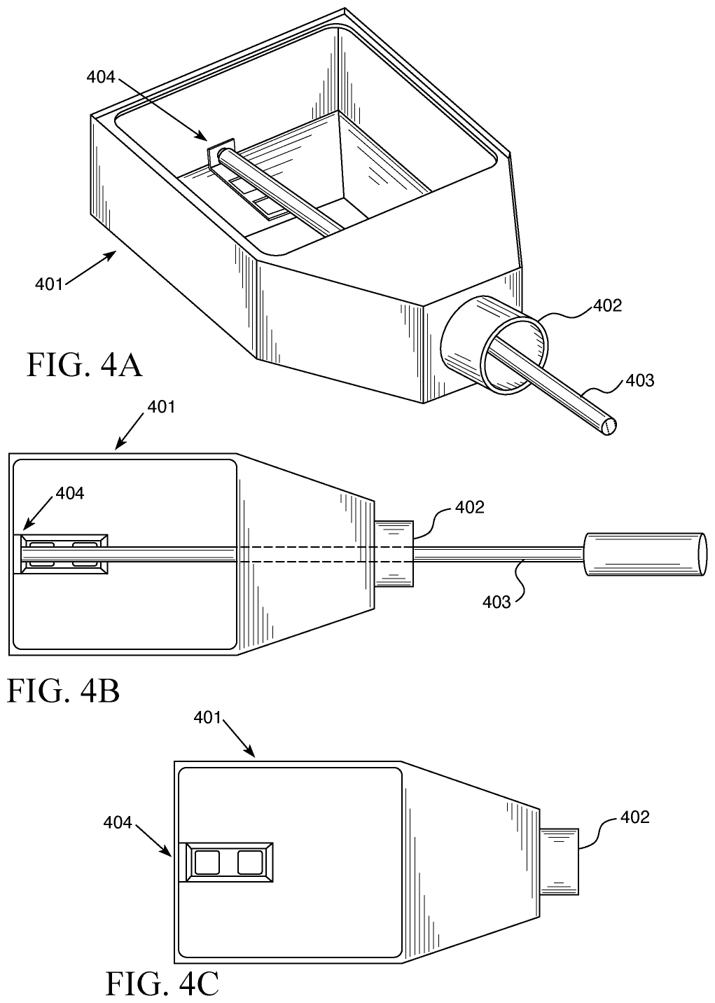

Referring to FIG. 4A, a method for inserting a sensor frame 404 into a cell culture flask 401 comprises inserting the sensor frame 404 while the sensor frame 404 is removably secured to the distal end of the sensor frame insertion apparatus 403 into the mouth 402 of a cell culture flask 401. In an aspect, the sensor frame 404 has an adhesive sheet adhered to the underside thereof.

In an aspect, the method comprises a step of applying pressure to a surface of the interior of the cell culture flask 401 with the sensor frame 404 and sensor frame insertion apparatus 403 so as to cause the adhesive sheet to adhere to an interior surface of the cell culture flask 401 so as to securely adhere the sensor frame 404 to the interior of the cell culture flask 401.

In an aspect, any cell culture flask may be used.

In another aspect, the cell culture flask may be a T-flask, an M-flask, an Erlenmeyer flask, or the like.

In an aspect, the cell culture flask may be of any volume, such as 50 mL, 75 mL, 100 mL, 125 mL, 150 mL, 175 mL, 200 mL, 250 mL, 300 mL, 350 mL, 400 mL, 450 mL, 500 mL, 100 mL, and so on.

FIG. 4B depicts a top-down view of a method for inserting a sensor frame 404 into a cell culture flask 401.

FIG. 4C depicts a top-down view of a sensor frame having been inserted into a cell culture flask, the sensor insertion apparatus having been unscrewed from the sensor frame after the adhesive sheet has adhered the underside of the sensor frame to a flat surface of the interior of the cell culture flask.

A sensor frame insertion apparatus described herein may, in some embodiments, have a means of adjusting the length of the elongated shaft which is permitted to enter the interior of a cell culture flask.

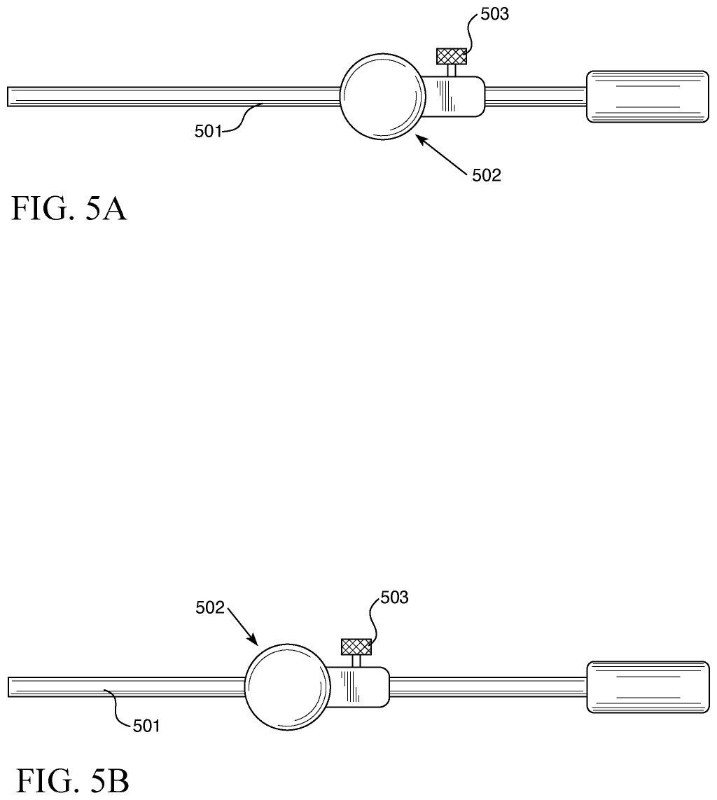

Referring to FIG. 5A, some embodiments of a sensor frame insertion apparatus may comprise an adjustable ball stop 502 positioned along the elongated shaft portion 501.

The adjustable ball stop 502 or other type of stopper may be moveable along the length of the elongated shaft portion 501 via friction.

In other embodiments, such as the one depicted in FIG. 5A, the adjustable ball stop 502 or other type of stopper may be movable along the length of the elongated shaft portion 501 via a thumb screw 503 or other tightening mechanism which is able to be loosened and tightened with finger/grip strength and without the need for additional tools or implements.

FIG. 5B shows the adjustable ball stopper positioned at a different position along the elongated shaft portion 501, the adjustable ball stop 502 having been moved by loosening the thumb screw 503, sliding the adjustable ball stop 502 along the elongated shaft portion 501 and re-tightening the thumb screw 503 when the adjustable ball stop 502 is in the desired position.

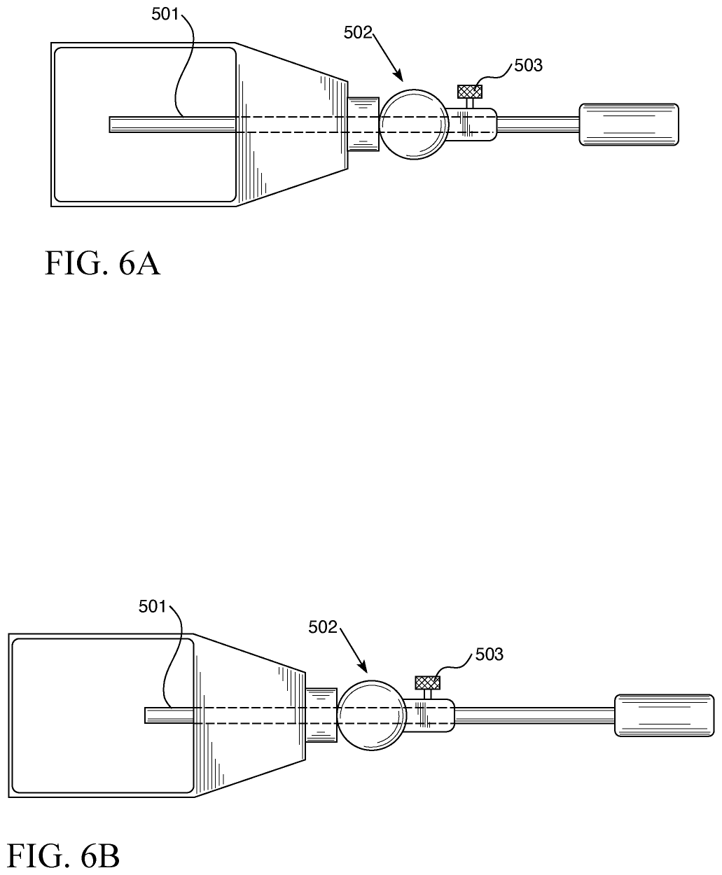

FIGS. 6A and 6B depict an exemplary sensor frame insertion apparatus having an adjustable ball stop positioned along the elongated shaft portion which has been inserted into a flask, wherein in FIGS. 6A and 6B the adjustable ball stopper is positioned at different positions along the elongated shaft portion, permitting different desired lengths of the elongated shaft portion to be inserted into the flask.

In an aspect, a ball stop, adjustable ball stop, or other type of stopper allows a user to insert the sensor frame insertion apparatus into a cell culture flask without fear of the apparatus jostling or otherwise moving in an undesired manner that would cause the apparatus to make contact with an interior surface of the cell culture flask that is desired to remain sterile or otherwise uncontaminated.

In certain embodiments, the elongated shaft portion may be telescoping to allow a user to set the elongated shaft portion to a desired length.

In an aspect, the cell culture flask is preferably placed on a detector system such that the sensors are operably connected to the detector system for measuring pH and/or dissolved gas concentrations, such as oxygen or carbon dioxide, or other measurable variables.

Embodiments can relate to a cell culture incubator system having sensors configured to be placed, in exemplary embodiments, within a flask described herein or, in other embodiments, any suitable reaction vessel selected by a person of skill in the art. The sensors can be configured so that when inserted via the sensor frame and sensor frame insertion apparatus, the sensors are positioned within media covering the cells.

A detector system or reader (e.g., a BioCoaster) may be placed outside but adjacent the vessel to read the sensor so as to detect changes in dissolved oxygen and pH.

The sensor insert is used to determine if the incubator environment is has too much carbon dioxide and is therefore trending towards hypoxia and/or acidity, or has too much dissolved oxygen and is therefore trending toward oxygen toxicity of the cell nutrient media.

Other conditions that can be determined may include the onset of apoptosis, growth toward equilibrium, deviations from equilibrium, etc.

Exemplary embodiments of sensor inserts for use in embodiments described herein are provided in U.S. Provisional Application No. 62/896,211, filed on Sep. 5, 2019. The entire contents of U.S. Provisional Application No. 62/896,211 are incorporated herein by reference.

Embodiments can relate to a cell culture incubator system having BioCoaster configured to be placed, in exemplary embodiments, under a flask described herein and/or used in conjunction or combination with a shake flask system described herein or, in other embodiments, any suitable reaction vessel selected by a person of skill in the art.

The BioCoaster can be configured so that when a sensor frame housing sensors adhered to an adhesive sheet is inserted into a flask or reaction vessel, the sensor is positioned within media covering the cells and above or adjacent to the BioCoaster. The BioCoaster is placed outside but adjacent the vessel to read the sensor so as to detect changes in dissolved oxygen and pH. Other conditions that can be determined may include the onset of apoptosis, growth toward equilibrium, deviations from equilibrium, etc.

Exemplary embodiments of detectors, e.g. BioCoasters, for use in embodiments described herein are provided in U.S. Pat. No. 10,379,047, issued on Aug. 13, 2019. The entire contents of U.S. Pat. No. 10,379,047 are incorporated herein by reference.

In an aspect, the sensors or sensor patches are to be aligned with a separate, external sensor-reading device. In certain embodiments, the sensor-reading device is a BioCoaster. Embodiments using different sensor-reading devices are hereby contemplated, so long as said sensor-reading devices are compatible with the sensor-spots housed on the adhesive sheet adhered to the underside of the sensor frame.

Various embodiments of the present disclosure can be described in terms of the example sensor insert described herein; however, other embodiments of the sensor insert, along with other flask and coaster (e.g., BioCoaster) embodiments, can be used.

In some embodiments, the cell culture flask is preferably placed on a rocker or shaker.

In an aspect, any suitable flask rocker or flask shaker device or mechanism may be used.

In some embodiments, the cell culture flask is placed on a detector system which is in turn placed on a rocker or shaker.

In other embodiments, the cell culture flask is placed on a rocker or shaker without the detector system. In some embodiments of the method described herein, the cell culture is rocked or shaken before or after being placed on the detector system in consecutive steps in any order, or in alternating steps.

It will be apparent to those skilled in the art that numerous modifications and variations of the described examples and embodiments are possible in light of the above teachings of the disclosure.

The disclosed examples and embodiments described herein and set forth in the accompanying figures are presented for purposes of illustration only. Other alternate embodiments may include some or all of the features disclosed herein.

Therefore, it is the intent to cover all such modifications and alternate embodiments as may come within the true scope of this invention, which is to be given the full breadth thereof.

Additionally, the disclosure of a range of values is a disclosure of every numerical value within that range, including the end points.

What is claimed:

1. An apparatus for use in cell culturing comprising:
an elongated shaft having a proximal end and a distal end;
a grippable portion at the proximal end of the elongated shaft;
a threaded portion at the distal end of the elongated shaft; and
a sensor frame having a threaded hole;
wherein the threaded portion at the distal end of the elongated shaft is configured to be removably screwed into the threaded hole of the sensor frame.

2. The apparatus according to claim 1, further comprising a stopper positioned along the elongated shaft.

3. The apparatus according to claim 2, wherein the stopper is a ball stop.

4. The apparatus according to claim 2, wherein the stopper is adjustably positioned along the elongated shaft.

5. The apparatus according to claim 1, wherein the elongated shaft is of an adjustable length.

6. The apparatus according to claim 4, wherein the stopper comprises a thumb screw.

7. The apparatus according to claim 1, wherein the sensor frame has an angled portion and wherein the threaded hole is located on the angled portion.

8. The apparatus according to claim 1, wherein the sensor frame does not have an angled portion.

9. A system for culturing cells comprising:
a flask;
a detector system;
at least one optical sensor operably connected to the detector system; and
an apparatus comprising an elongated shaft having a proximal end and a distal end, a grippable portion at the proximal end of the elongated shaft, a threaded portion at the distal end of the elongated shaft, and a sensor frame having a threaded hole;
wherein the threaded portion at the distal end of the elongated shaft is configured to be removably screwed into the threaded hole of the sensor frame; and
wherein the at least one optical sensor is positioned within or on the sensor frame.

10. The system according to claim 9, further comprising a flask rocker or shaker.

11. The system according to claim 9, wherein the sensor frame is attachable to an interior surface of the flask.

12. The system according to claim 11, wherein the sensor frame comprises an adhesive sheet applied to an underside of the sensor frame.

13. The system according to claim 12, wherein the at least one optical sensor is adhered to an upper side of the adhesive sheet.

14. The system according to claim 9, wherein the flask is a T-flask.

15. A method for culturing cells comprising:
placing an adhesive sheet on an underside of a sensor frame having a threaded hole;
placing at least one optical sensor on or within the sensor frame;
inserting the sensor frame into a flask such that the adhesive sheet makes contact with an interior surface of the flask and adheres to the interior surface; and
placing the flask on or against a detector system; and
culturing cells in the flask;
wherein the at least one optical sensor is operably connected to the detector system;
wherein the inserting is carried out using an apparatus; and
wherein the apparatus comprises an elongated shaft having a proximal end and a distal end, a grippable portion at the proximal end of the elongated shaft, a threaded portion at the distal end of the elongated shaft, wherein the threaded portion at the distal end of the elongated shaft is configured to be removably screwed into the threaded hole of the sensor frame.

16. The method according to claim 15, further comprising placing the flask on a shaker or rocker.

17. The method according to claim 15, wherein the sensor frame has an angled portion and wherein the threaded hole is located on the angled portion

18. The method according to claim 15, wherein the sensor frame does not have an angled portion.

19. The method according to claim 15, wherein the at least one optical sensor is adhered to an upper side of the adhesive sheet.

20. A sensor frame, comprising:
a body having one or more openings, the body having a first surface and a second surface;
a double-sided adhesive sheet disposed on the second surface;
a first film placed on the first surface and temporarily adhered to the body by making contact with the double-sided adhesive sheet via the one or more openings; and
a second film placed on the second surface and temporarily adhered to the body via the double-sided adhesive sheet;
wherein the first film is removable to facilitate attaching at least one optical sensor on the double-sided adhesive sheet via the one or more openings; and
wherein the second film is removable to expose the adhesive and facilitate attaching the sensor frame to an inner surface of a flask.
